(19) United States
(12) Patent Application Publication
(10) Pub. No.: US 2016/0235623 A1
(43) Pub. Date: Aug. 18, 2016

(54) Invasive Intermittent Negative Pressure Ventilation

(71) Applicant: Michael Mirzoyan, Las Vegas, NV (US)

(72) Inventor: Michael Mirzoyan, Las Vegas, NV (US)

(21) Appl. No.: 14/622,958

(22) Filed: Feb. 16, 2015

(51) Int. Cl. A61H 31/02; A61M 5/14; A61M 39/02; A61M 39/24; A61M 16/00; A61M 16/06

(52) U.S. Cl. CPC A61H 31/02; A61M 16/0057; A61M 16/0666; A61M 39/0208; A61M 39/24; A61M 5/14; A61M 2230/40; A61M 2039/0252; A61M 2039/0223

(57) ABSTRACT

The present invention introduces novel invasive intermittent negative pressure ventilation for treatment of adults ventilator dependent respiratory failure (VDRF) in various advanced neuromuscular disorders (NMDs). It's a combination of invasive intermittent negative pressure ventilator (IINPV) and continues positive airway pressure (CPAP) into transportable ventilator. Implementation of invasive negative pressure ventilation is via chest tubes to each pleural space, and CPAP is applied via nasal mask or nasal prongs. The IINPV's initiation of breath is patient-triggered via a flow sensor incorporated in CPAP nasal mask/nasal prongs. This novel concept of VDRF management in advanced NMDs will prove to be superior over present modalities of treatment in many aspects which include but not limited to avoidance of tracheostomies, reduction of recurrent respiratory infection, greater patient's independence and quality of life, preserved ability to speak and clear airways spontaneously, liberation from positive pressure mechanical ventilation etc.

### FIELD OF THE INVENTION

The present invention relates generally to the field of respiratory enhancement, and in particular to a concept of invasive (intrathoracic, intrapleural) intermittent negative pressure ventilation (IINPV) and methods for providing an adult patient with invasive (intrathoracic, intrapleural) negative pressure ventilation. More specifically, the invention relates to devices for pulmonary ventilation at negative and positive pressures, useful for aiding respiration of patients suffering from ventilator dependent respiratory failure (VDRF) due to advanced neuromuscular disorders.

### BACKGROUND OF THE INVENTION

Patients affected by respiratory insufficiency often require assisted ventilation to facilitate pulmonary ventilation and consequent respiratory air exchange. In prevalent number of neuromuscular illnesses like Myasthenia Gravis, Guillain-Barre syndrome, Amyotrophic Lateral Sclerosis, Poliomyelitis, Muscular Dystrophies, high level spinal cord injury, Diaphragmatic Paralysis etc., adult patients become dependent on a respirator due to development of respiratory failure. Presently the management of the respiratory failure in these patients commonly is requiring a surgical tracheostomy in conjunction with positive pressure mechanical ventilation.

Nowadays, there are a growing number of people in the category of advanced neuromuscular disorders who refuse tracheostomies placements and succumb to the respiratory failure. Other contemporary used modalities in the treatment of adults ventilator dependent respiratory failure in advanced neuromuscular disorders are diaphragmatic nerve pacing, noninvasive positive pressure ventilation, noninvasive extrathoracic negative pressure ventilation are commonly ineffective in long-term management of these patients due to variety of factors.

With the aim to provide assistance for adult patients affected by ventilator dependent respiratory failure in advanced neuromuscular disorders, several devices have been provided in the past which were based on the application on the patient's thorax and abdomen of a closed box delimiting a sealed gap between said box and the patient's body: an noninvasive intermittent negative and/or negative and positive pressure is applied to said gap.

The noninvasive (extrathoracic) negative pressure applied outside the patient's thorax determines a negative pressure inside the airways thus provoking an inspired air flow, while a positive pressure, on the contrary, provokes expiration airflow.

As an example of such devices for assisting patients affected by respiratory insufficiency there may be cited those of U.S. Pat. No. 5,474,533; U.S. Pat. No. 8,408,204; U.S. Pat. Appl. No. 20070199566 A1 and U.S. Pat. Appl. No. 20090171256 A1.

A further device for assisting patients affected by chronic respiratory insufficiency is represented by the “chest cuirasses”, which consists of a plastic rigid shell-shaped bell which embraces, while sealing, the anterior surface of the thorax and a portion of the patient's abdomen, and of a pump to create negative and positive pressures. This device, light and portable, may be also used at home, but it requires that the shell-shaped bell be tailored to the single thorax to avoid air leakages. Moreover the pressures exerted around the body of the patient are uncomfortable after one or two hours of operation and can even cause thorax and dorsal pains and skin lesions.

Yet a further device useful for helping patients affected by respiratory insufficiency is the “poncho”. It is formed by a nylon covering with a rigid, flat portion, supporting the back, and a rigid shell-shaped portion anchored to the flat portion; inside the device are located the thorax and the abdomen of the patient. The poncho is then sealed around the neck, the wrists, the pelvis and the ankles of the patient. A negative pressure is intermittently applied on the surface of the thorax and the abdomen. Although poncho is better tolerated than the cuirasses′, it may provoke back pains because of its rigid flat dorsal portion and also, sometimes, the patients complain of coldness.

In the last years of the last century non-invasive positive pressure ventilation was put on the market. These devices are relatively small, light and portable. But there is a limitation with these devices in its use in case of ventilator dependent respiratory failure due to advanced neuromuscular disorders.

It is therefore desirable to have available a negative-positive pressure ventilation device which is transportable, usable during normal life and easy to be used by patients and by medical doctors.

In contrast present invention introduces a novel invasive (intrathoracic, intrapleural) application of intermittent negative pressure ventilation for the treatment of chronic ventilator dependent respiratory failure in various advanced neuromuscular illnesses. It is proposed here as a combination of invasive (intrathoracic, intrapleural) intermittent negative pressure ventilator (IINPV) and continues positive airway pressure (CPAP) into one transportable ventilator.

### SUMMARY OF THE INVENTION

In the view of the foregoing disadvantages inherent in the known types of noninvasive (extrathoracic) negative intermittent pressure ventilation devices now present in the prior art, the present invention provides a novel combination of invasive (intrathoracic, intrapleural) intermittent negative pressure ventilator (IINPV) and continues positive airway pressure (CPAP) into one transportable ventilator useful for assisting respiration of patients affected by ventilator dependent respiratory failure in various advanced neuromuscular disorders.

As such, the general purpose of the present invention, which will be described subsequently in greater detail, is to introduce a novel concept of invasive (intrathoracic, intrapleural) negative pressure ventilation which has all the advantages of the prior art and none of the disadvantages.

The invasive (intrathoracic, intrapleural) intermittent negative pressure ventilation further includes two portable IINPV units and one CPAP unit within one device. Also, the initiation of the IINPV breath is triggered via a flow sensor incorporated in the CPAP nasal mask/nasal prongs.

The invasive (intrathoracic, intrapleural) intermittent negative pressure ventilator further connected to the patient's intrapleural spaces by the means of two non-compressible chest tubes, each one on the opposite sides (left and right hemi-thoraces) and in the inferior (anterolateral vs mediolateral vs posterior-lateral) aspects of the chest.

The invasive (intrathoracic, intrapleural) intermittent negative pressure ventilator also includes a plastic cylindrical tip at the intrapleural end of the chest tube in which tip is fenestrated with holes.

The invasive (intrathoracic, intrapleural) intermittent negative pressure ventilator also includes a one-way valve inside the chest tube, directed (open) outside of the chest and provided with the least resistance to opening negative pressure.

The invasive (intrathoracic, intrapleural) intermittent negative pressure ventilator also includes a circular rubber cuff at the intrapleural end of the chest tube which is further connected with a spring-valve outside the chest via a small-diameter channel within the wall of the chest tube.

In another aspect, method includes the intrapleural insertion of the chest tubes which is carried out by minimally invasive ways via skin incisions. After the chest tube is inserted, the cuff is expanded with instillation of sterile normal saline and the chest tube is pulled back by an operator until resistance is felt. The expanded and pulled back circular cuff will provide a seal at the insertion point inside the chest and prevent further outward displacement of the chest tube during long-term management. After the cuff is expanded and the chest tube pulled back, the skin incision around the chest tube is sutured, providing a seal around the chest tube and securing the placement. Addition of synthetic/rubber flange around the chest tube on the skin at the insertion site may further improve the security of the tube placement by preventing or minimizing inward dislodgement of the chest tube.

In addition the main characteristics of the ventilator dependent respiratory failure management in advanced neuromuscular disorders consists a novel aspects which include but not limited to avoidance of tracheostomies, reduction of recurrent lower and upper airway infections, greater degree of independence, improved quality of life, no need for frequent airway suctioning, preserved ability to speak and clear airways spontaneously, liberation from positive pressure mechanical ventilation etc.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

These together with other objects of the invention, along with the various features of novelty which characterize the invention, are pointed out with particularity in the disclosure. For a better understanding of the invention, its operating advantages and the specific objects attained by its uses, reference should be had to the accompanying drawings and descriptive matter in which there are illustrated preferred embodiments of the invention.

### BRIEF DESCRIPTION OF THE FIGURES

The invention will be better understood and objects other than those set forth above will become apparent when consideration is given to the following detailed description thereof. Such description makes reference to the annexed drawings wherein:

FIG. 1 illustrates combination of invasive (intrathoracic, intrapleural) intermittent negative pressure ventilator (IINPV) and continues positive airway pressure (CPAP) into one transportable ventilator.

FIG. 2 shows detailed view of cylindrical plastic tip of the chest tube used in device depicted in FIG. 1.

FIG. 3 is a detailed view of different embodiment like, circular rubber cuff, one way valve, cylindrical tip, and chest tube which is used in combination in device depicted in FIG. 1.

### DETAILED DESCRIPTION OF THE INVENTION

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that the embodiments may be combined, or that other embodiments may be utilized and that structural, logical and electrical changes may be made without departing from the spirit and scope of the present invention. The following detailed description is, therefore, not to be taken in a limiting sense, and the scope of the present invention is defined by the appended claims and their equivalents.

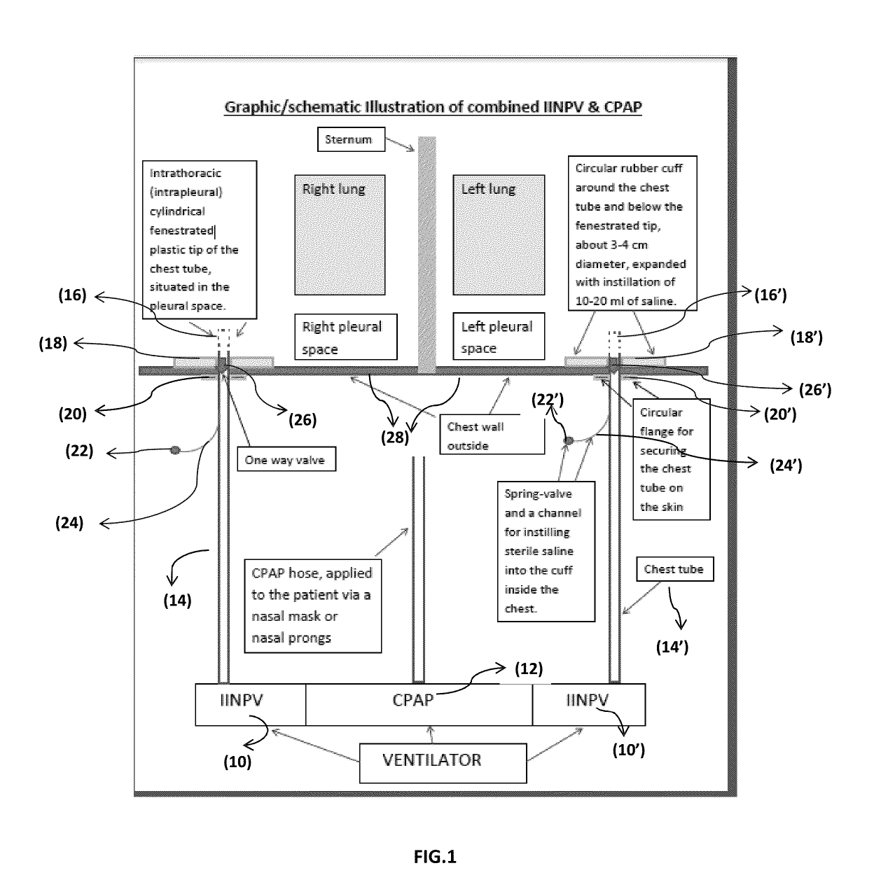

On referring FIG. 1 which shows the invasive (intrathoracic, intrapleural) intermittent negative pressure ventilator (IINPV) as a whole, comprising of: two separate portable IIPNV units (10), (10′) in order to apply negative pressure separately in each plural space; one CPAP unit with nasal mask/nasal prongs (12); two non-compressible chest tubes (14),(14′); at the intrapleural end of the chest tubes a plastic cylindrical tip (16), (16′); a circular rubber cuff (18), (18′) on the external surface of the intrapleural end of the chest tube; a spring-valve (22), (22′) and its channel (24), (24′) outside the chest wall (28) within the wall of the chest tube; one-way valve (26) (26′) inside the chest tube, directed outside of the chest.

The invasive (intrathoracic, intrapleural) intermittent negative pressure ventilator (IINPV) proposed here is a patient triggered, time cycled and pressure limited, with negative pressure limit is set in the range of −10 to −20 cm H₂O and applicable with the help of chest tubes to each pleural space separately (left and right hemi-thoraces). This separate (left and right pleural spaces) application of invasive (intrathoracic, intrapleural) negative pressure ventilation may require operation of two separate portable IINPV units (10), (10′) within one device in order to apply negative pressure in each pleural space via respective chest tubes (14),(14′). The initiation of IINPV breath is triggered via a flow sensor incorporated in the CPAP nasal mask/nasal prongs (12). CPAP is set in the range +5 to +10 cm H2O and applied to a patient via nasal mask or nasal prongs to provide pneumatic upper airway splint in order to facilitate breaths delivered by IINPV. Also, IINPV has a backup rate set in the range of 6-12 breaths per minute, in case if the patient fails to trigger IINPV, i.e. prolonged apnea. Consequently, the lungs ventilation will be provided by generation of intrapleural intermittent negative pressure, therefore effectively substituting weakened diaphragmatic function.

The IINPV device is connected to the patient's intrapleural spaces by the means of two non-compressible chest tubes (14), (14′) about 0.5-1 cm in diameter, each one on the opposite sides (left and right hemi-thoraces) and in the inferior (anterolateral vs mediolateral vs posterior-lateral) aspects of the chest. The intrapleural insertion of the chest tubes carried out in the minimally invasive ways via skin incisions and in the tunneled manner to minimize risk for ascending infection.

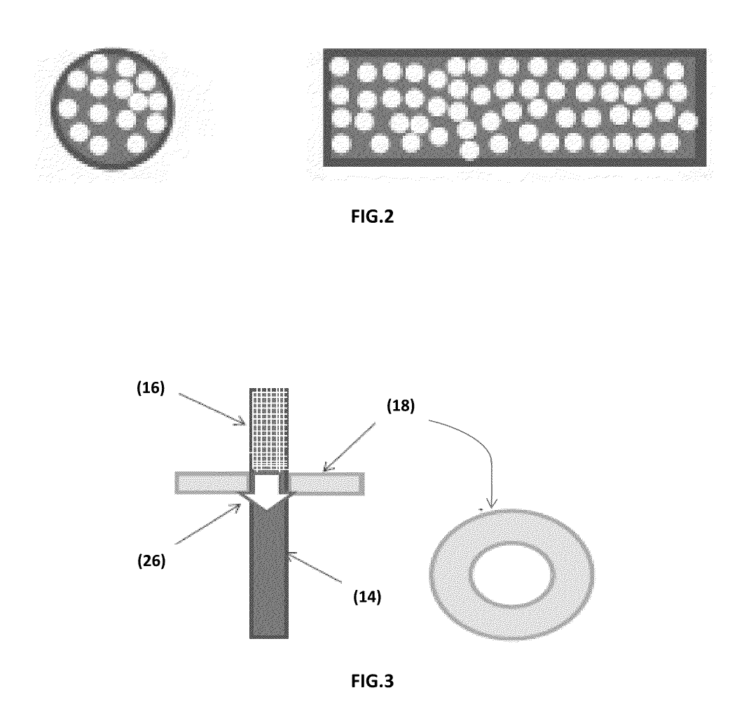

On referring FIG. 2, the intrapleural end of the chest tubes (14), (14′) has a plastic cylindrical tip about 2-3 cm height and 1 cm in diameter. The entire perimeter including the end of the cylindrical plastic tip (16), (16′) of the chest tube is fenestrated with 3-4 mm holes. It is preferred that the plastic tip of the chest tube to be drug (anti-proliferative) eluting to prevent or minimize formation of pleural adhesions around the inserted portion of the chest tube.

The fenestrated cylindrical plastic tip (16), (16′) of the chest tube (inside the chest) transitions into non-fenestrated synthetic non-compressible chest tube which constitutes predominant length of the tube and mostly situated outside of the chest. At the transitional point, when the fenestrated cylindrical plastic tip (16), (16′) ends and non-fenestrated synthetic non-compressible chest tube begins, there is a circular rubber cuff (18), (18′) on the external surface of the intrapleural end of the chest tube.

According to FIG. 3 a circular rubber cuff (18), (18′) is connected with a spring-valve (22), (22′) outside the chest via a small-diameter channel (24), (24′) within the wall of the chest tube. The syringe instilled around 10-20 ml of sterile normal saline via the spring-valve (22), (22′) into the cuff and expands the cuff to about 3-4 cm in diameter and 0.5-1 cm thick around the intrapleural end of the chest tube. In the opposing manner, the saline is removed when needed to deflate the cuff (e.g. removal or reposition of the chest tube) with the use of the syringe to aspirate saline through the spring-valve. While the cuff is deflated, it should not significantly affect the diameter of the chest tube and therefore, will not impact on the insertion of the chest tube.

According to another embodiment the above mentioned transitional zone, i.e. when the fenestrated cylindrical plastic tip ends and non-fenestrated synthetic non-compressible chest tube begins, there is a one-way valve (26) (26′) inside the chest tube, directed (open) outside of the chest (28) and provided with the least resistance to opening negative pressure.

After the chest tube (14), (14′) is inserted, the cuff (18), (18′) is expanded with instillation of sterile normal saline and the chest tube is pulled back by an operator until resistance is felt. The expanded and pulled back circular cuff will provide a seal at the insertion point inside the chest and also prevent from further outward displacement of the chest tube during long-term management. After the cuff (18), (18′) is expanded and the chest tube (14), (14′) pulled back, the skin incision around the chest tube is sutured providing a seal around the chest tube and securing the placement. Addition of synthetic/rubber flange (20), (20′) around the chest tube on the skin at the insertion site may also improve the security of the tube placement by preventing or minimizing inward dislodgement of the chest tube.

Advantageously, new concept of invasive (intrathoracic, intrapleural) negative pressure ventilation and the device along the medical management described herein provide novel management of adults ventilator dependent respiratory failure in advanced neuromuscular illnesses may prove to be superior over present modalities in many aspects which include but not limited to avoidance of tracheostomies, reduction of recurrent lower and upper airway infections, greater degree of independence, improved quality of life, no need for frequent airway suctioning, preserved ability to speak and clear airways spontaneously, liberation from positive pressure mechanical ventilation etc.

It is to be understood that the above description is intended to be illustrative, and not restrictive. For example, the above-discussed embodiments may be used in combination with each other. Many other embodiments will be apparent to those of skill in the art upon reviewing the above description.

The benefits and advantages which may be provided by the present invention have been described above with regard to specific embodiments. These benefits and advantages, and any elements or limitations that may cause them to occur or to become more pronounced are not to be construed as critical, required, or essential features of any or all of the embodiments.

While the present invention has been described with reference to particular embodiments, it should be understood that the embodiments are illustrative and that the scope of the invention is not limited to these embodiments. Many variations, modifications, additions and improvements to the embodiments described above are possible. It is contemplated that these variations, modifications, additions and improvements fall within the scope of the invention.

What is to be claimed:

1. An invasive (intrathoracic, intrapleural) intermittent negative pressure ventilation.

2. A combined invasive (intrathoracic, intrapleural) intermittent negative pressure ventilator (IINPV) and continues positive airway pressure (CPAP) unit into one transportable ventilator, wherein said device comprises:
at least one chest tube for application of said intrapleural intermittent negative pressure ventilation;
at least one IINPV unit;
at least one CPAP unit;
at least one flow sensor incorporated in CPAP nasal mask/nasal prongs;
at least one fenestrated plastic cylindrical tip with at least one hole at the intrapleural end of each said chest tube inside the chest;
at least one circular rubber cuff on the external surface of the intrapleural end of each said chest tube;
at least one, one-way valve opening outside of the chest in the direction of said IINPV unit;
at least one spring-valve outside the chest with a channel within the wall of each said chest tube;

3. The combined transportable IINPV and CPAP ventilator of claim 2, wherein said negative pressure is applicable with the help of said chest tubes to each pleural space of lungs (left and right hemi-thoraces) separately.

4. The combined transportable IINPV and CPAP ventilator of claim 2, wherein said IINPV has a backup rate set in the range of 6-12 breaths per minute.

5. The combined transportable IINPV and CPAP ventilator of claim 2, wherein said IINPV breath is triggered via said flow sensor incorporated in the said CPAP nasal mask/nasal prongs.

6. The combined transportable IINPV and CPAP ventilator of claim 2, wherein said device is further connected to the patient's intrapleural spaces by the means of said non-compressible chest tubes.

7. The combined transportable IINPV and CPAP ventilator of claim 2, wherein said intrapleural cylindrical plastic tip of each said chest tube is fenestrated with holes.

8. The combined transportable IINPV and CPAP ventilator of claim 2, wherein said circular rubber cuff is connected with a said spring-valve outside the chest via a small-diameter channel within the wall of each said chest tube.

9. The combined transportable IINPV and CPAP ventilator of claim 2, wherein said one-way valve inside each said chest tube, directed (open) outside of the chest and provided with the least resistance to the opening negative pressure.

10. An invasive (intrathoracic, intrapleural) intermittent negative pressure ventilator (IINPV) and continues positive airway pressure (CPAP) into one transportable ventilator, wherein said device comprises:
a portable IINPV units within said device in order to apply negative pressure in each pleural space via respective chest tubes;
said IINPV has a backup rate set in the range of 6-12 breaths per minute which prevents prolonged apneas;
said IINPV is connected to the patient's intrapleural spaces by the means of two non-compressible chest tubes;
said chest tubes inside the chest has a intrathoracic (intrapleural) cylindrical fenestrated plastic tip;
inside each said chest tube there is a one-way valve open outside the chest;
at the transitional point wherein said fenestrated cylindrical plastic tip ends and non-fenestrated synthetic non-compressible chest tube begins, there is a circular rubber cuff on the external surface of each intrapleural end of said chest tube;
said circular rubber cuff is connected with a spring-valve outside the chest via a small-diameter channel within the wall of each said chest tube;
said circular rubber cuff is expanded and deflated at the instillation and removal of sterile normal saline with the help of syringe;

11. The combined transportable IINPV and CPAP ventilator of claim 10, wherein said negative pressure limit is set in the range of −10 to −20 cm H₂O.

12. The combined transportable IINPV and CPAP ventilator of claim 10, wherein said CPAP is set in the range +5 to +10 cm H₂O which is applied to the patient via nasal mask or nasal prongs.

13. The combined transportable IINPV and CPAP ventilator of claim 10, wherein said non-compressible chest tubes are about 0.5-1 cm in diameter.

14. The combined transportable IINPV and CPAP ventilator of claim 10, wherein said intrathoracic (intrapleural) cylindrical fenestrated plastic tip is 2-3 cm in height and 1 cm in diameter.

15. The combined transportable IINPV and CPAP ventilator of claim 14, wherein said intrapleural cylindrical plastic tip of said chest tube is fenestrated with 3-4 mm holes.

16. The combined transportable IINPV and CPAP ventilator of claim 14, wherein said intrapleural cylindrical plastic tip of said chest tube is drug eluting to prevent formation of pleural adhesions around the inserted portion of said chest tube.

17. The combined transportable IINPV and CPAP ventilator of claim 10, wherein said 10-20 ml of sterile normal saline is instilled via said spring-valve into said circular rubber cuff.

18. The combined transportable IINPV and CPAP ventilator of claim 10, wherein said circular rubber cuff is expanded approximately 3-4 cm in diameter and 0.5-1 cm thick around each intrapleural end of said chest tube.

19. The combined transportable IINPV and CPAP ventilator of claim 10, wherein said one-way valve in said chest tube is directed (open) outside of the chest.

20. A medical method for treating a person with combined IINPV and CPAP device comprising:
providing intrapleural insertion of said chest tubes carried out in the minimally invasive ways via skin incisions and in the tunneled manner;
at intrapleural end of said chest tubes inside the chest has said fenestrated cylindrical plastic tip;
at transitional point where said fenestrated cylindrical plastic tip ends and non-fenestrated synthetic non-compressible said chest tube begins is said circular rubber cuff which is adherent to the external surface of the intrapleural end of said chest tube;
said rubber cuff is connected with said spring-valve outside the chest via a small-diameter channel within the wall of each said chest tube;
through said spring-valve sterile normal saline is instilled into the said rubber cuff which expands said cuff and provides a seal at the insertion point inside the chest;

21. The medical method for treating a person with combined IINPV and CPAP device of claim 20, wherein said IINPV breath is triggered via a flow sensor incorporated in the CPAP nasal mask/nasal prongs.

22. The medical method for treating a person with combined IINPV and CPAP device of claim 20, wherein said chest tubes are inserted intrapleurally into left and right hemi-thoraces and in the inferior (anterolateral vs mediolateral vs posterior-lateral) aspects of the chest.

23. The medical method for treating a person with combined IINPV and CPAP device of claim 20, wherein said fenestrated cylindrical plastic tip of said chest tube is drug eluting to prevent or minimize formation of pleural adhesions around the inserted portion of said chest tube.
